(19) United States
(12) Patent Application Publication
(10) Pub. No.: US 2017/0326336 A1
(43) Pub. Date: Nov. 16, 2017

(54) BALLOON CATHETER WITH RADIOPAQUE MARKER

(71) Applicant: BIOTRONIK AG, Buelach (CH)

(72) Inventor: Amir Fargahi, Buelach (CH)

(21) Appl. No.: 15/583,573

(22) Filed: May 1, 2017

(30) Foreign Application Priority Data
May 12, 2016 (DE) 10 2016 108 783.1

(51) Int. Cl. A61M 25/01; A61F 2/958; A61M 25/10

(52) U.S. Cl. CPC A61M 25/0108; A61M 25/0125; A61F 2/958; A61F 2250/0098; A61M 2025/1079

(57) ABSTRACT

A balloon catheter includes an expandable balloon and an inner shaft arranged in the balloon. At least one marker forms a radiopaque material. The radiopaque material is formed and/or in a polymer carrier, and the polymer carrier is fastened to the inner shaft. A simple, reliable and economical method for producing a balloon catheter is also provided.

### PRIORITY CLAIM

This application claims priority under 35 U.S.C. §119 and all applicable statutes and treaties from prior German Application DE 10 2016 108 783.1, filed May 12, 2016.

### FIELD OF THE INVENTION

A field of the invention is balloon catheters. Balloon catheters are typically employed during minimally invasive surgical procedures for the widening or re-opening of constricted or closed blood vessels.

### BACKGROUND

A balloon catheter is a tool for the minimally invasive widening or re-opening of constricted or closed blood vessels, often caused by calcifications in the vessel. In the case of percutaneous transluminal coronary angioplasty (PTCA), a balloon catheter is usually inserted in the constricted point from the groin via a guide wire, and then is subjected to a pressure of several bar. The pressure dilates the balloon in the vessel and presses from inside against the vessel wall or the calcifications. It is thus often possible to expand the constricted point again. In order to enable an accurate insertion and positioning of the catheter in the body, the position of the balloon is monitored by an x-ray apparatus. For this purpose, the balloon catheter has markers (markings) made of a radiopaque material. By way of example, a marker is attached at both ends of the balloon, which has the additional advantage that the alignment of the balloon catheter can also be monitored, in addition to the position.

Typical balloon catheters have an expandable balloon, which surrounds an inner shaft. Round platinum or gold sleeves are usually used as radiopaque markers. In order to fasten these to the inner shaft of the balloon catheter, a swaging process is usually used. The fastening of the marker by swaging has proven to be unsatisfactory for a number of reasons.

Sharp edges can form at the marker as a result of the swaging and can lead to a perforation of the balloon and therefore to a balloon that is not functional. Even with the smallest amount of damage in the wall, the application of pressure can be impaired, such that the balloon either cannot be fully dilated or cannot act on the vessel wall with the necessary pressure. Another problem is that the marker can shift during the fastening process as force is applied to the marker as a result of the swaging. Thus, neither the precise position of a marker nor the distance between the proximal and the distal marker can be maintained in an exact manner. This can lead to such large deviations that a balloon catheter is not useful. The application of force during swaging can also cause an inner channel of the inner shaft serving to receive the guide wire to become constricted. In addition, an automation of the swaging is difficult. The production by means of swaging is thus time-consuming, costly, and the balloon catheter produced as a result often does not satisfy the specified quality requirements. It is additionally disadvantageous that metal sleeves of this type have a great rigidity, which hinders the movement of the catheter as it is introduced into the vessel to be treated.

Alternatively, polymer markers which for example consist of a polymer material, such as nylon, urethane or thermoplastic elastomers, which is filled with tungsten can be used. A disadvantage of these polymer markers is their great thickness, which is necessary for the sufficient radiopacity and which increases the cross-section of a balloon catheter. An automation of the production of such polymer markers is also not practical.

### SUMMARY OF THE INVENTION

The invention relates to a balloon catheter having an expandable balloon, an inner shaft arranged in the balloon, and at least one marker, wherein the at least one marker comprises a radiopaque material. The invention additionally relates to a method for producing such a balloon catheter. A balloon catheter of the invention has a high radiopacity, lacks any sharp edges at the x-ray markers, and provides x-ray markers arranged at an exact predetermined position. The invention also relates to a simple, reliable and economical method for producing a balloon catheter of this type.

A preferred embodiment is a balloon catheter having an expandable balloon. An inner shaft is arranged in the balloon. The balloon includes at least one marker. The at least one marker is a radiopaque material on and/or in a polymer carrier. The polymer carrier is fastened to the inner shaft. The polymer carrier can be sleeve-like, planar or strip-like, and can be curved at least in part around the inner shaft. The carrier preferably contains polyimide and/or LCP and/or has a thickness between 10 and 50 μm, preferably between 20 and 30 μm. the radiopaque material contains at least one material from the group comprising gold, tantalum, niobium, platinum and alloys of these materials and/or the coating has a thickness between 20 and 100 μm, preferably 40 and 60 μm. Preferred embodiments include a first marker is arranged in a first portion of the polymer carrier, and a second marker is arranged in a second portion of the polymer carrier, wherein the first marker and the second marker are arranged at a predetermined distance.

A preferred embodiment is a method for producing a balloon catheter with an inner shaft arranged in a balloon. One of a polymer carrier board, a polymer carrier strip, or a polymer carrier sleeve is provided. A radiopaque material in the form of at least one marker is applied to and/or introduced into the polymer carrier board, the polymer carrier strip, or the polymer carrier sleeve. The polymer carrier board, the polymer carrier strip, or the polymer carrier sleeve is then fastened on the inner shaft of the balloon catheter. The fastening is preferably conducted via a polymer carrier strip, which preferably has been separated from a polymer carrier board via a procedure wherein the polymer carrier strip is first fixed on the inner shaft, the inner shaft is then arranged within the balloon, and then the balloon, inner shaft and polymer strip are welded simultaneously to one another at their distal end. In preferred methods, the polymer carrier board has a longitudinal direction and a transverse direction at right angles thereto, in that the radiopaque material is provided in the form of at least two strips running in the transverse direction at a defined distance, and in that the polymer carrier strip is separated from the polymer carrier board in the longitudinal direction

### BRIEF DESCRIPTION OF THE DRAWINGS

Advantages and expedient features of the invention will also become clear from the following description of an exemplary embodiment provided with reference to the figures, in which:

FIG. 1 schematically shows a balloon catheter according to the invention in a cross-section;

FIG. 2A-2C schematically show the production of a polymer carrier strip in a view from above and in a view from the side;

FIG. 3 schematically shows a polymer carrier of a balloon catheter according to the invention which is embodied as a polymer carrier sleeve, in a perspective view from the side, and

FIG. 4A-4C schematically show the steps of producing a balloon catheter according to the invention in a view from the side and in a sectional illustration.

### DETAILED DESCRIPTION OF THE PREFERRED EMBODIMENTS

In accordance with the invention, the radiopaque material is a marker that can formed as a coating on and/or in a polymer carrier which is fastened to the inner shaft. The coating can consist completely or at least in part of the radiopaque material.

The radiopaque material can be arranged for example merely on the surface of the polymer carrier. Within the scope of the invention, however, it is also possible to apply the coating in the region of an indentation or recess in the polymer carrier. As a result, the thickness of the marker can be increased, which leads to an improved radiopacity. It is also possible that the radiopaque material diffuses at least in part into the polymer carrier during the application.

Due to the production process, a coating according to the invention on a polymer carrier does not have any sharp edges which could lead to damage of the balloon. It is thus ensured that the balloon can be reliably dilated when acted on by pressure, for example in the body of a patient. In addition, the risk of injuries occurring inside the body of a patient caused by sharp edges is eliminated. Compared to other methods, a marker according to the invention can be positioned in a substantially exact manner on the polymer carrier and therefore on the balloon catheter. This increases the accuracy with which the position of the balloon catheter can be determined on the basis of an x-ray image. In addition, it is avoided that the fastening of the marker to the inner shaft leads to a constriction of a channel within the inner shaft. Since the inner shaft is generally likewise manufactured from a polymer material, a further advantage of the solution according to the invention lies in the fact that the polymer carrier can be easily fastened to the inner shaft. Depending on the selection of the material of the polymer carrier, this, together with the at least one marker, additionally has a greater flexibility compared to a metal sleeve.

In a preferred embodiment of the invention a first marker is arranged in a first portion of the polymer carrier, and a second marker is arranged in a second portion of the polymer carrier. The first marker and the second marker here are arranged at a precisely defined distance from one another. As a result, in addition to the position of the balloon catheter, the orientation thereof can also be determined in an x-ray image. Due to the precisely defined distance, when monitoring the balloon catheter in the x-ray apparatus it is also possible to identify, in a precise manner, the length over which the vessel is widened by the balloon catheter. The distance between the markers is determined by way of example by the distance between the centrelines of the markers.

In a further preferred embodiment of the invention the polymer carrier is fastened by its proximal end to the inner shaft by means of a welded connection, preferably at individual points, and/or the polymer carrier is fastened by its distal end likewise to the inner shaft by means of a preferably peripheral, fluid-tight welded connection. The polymer carrier is particularly preferably jointly welded by its proximal end to the inner shaft and the outer shaft. The polymer carrier is particularly preferably fastened by its distal end by means of a simultaneous welding of balloon, inner shaft and a tip of the balloon catheter.

Alternatively to the welding, the polymer carrier can be fastened externally onto the inner shaft by means of an adhesively bonded connection, i.e. using an adhesive.

It is also advantageous if the polymer carrier is planar or strip-like and the polymer carrier is curved at least partially around the inner shaft in order to be fastened to the inner shaft.

The polymer carrier can have a width which corresponds to the periphery of the inner shaft, such that the polymer carrier is curved once around the inner shaft. A planar or strip-like polymer carrier can be easily produced and processed.

If the polymer carrier is strip-like, is fastened laterally to the inner shaft, and has merely a width which is much smaller than the periphery of the inner shaft, the inner shaft thus has a high flexibility, even after the fastening of the coated polymer carrier. The balloon catheter according to the invention can thus be guided more easily through narrow vessel passages.

In an alternative embodiment of the invention the polymer carrier is sleeve-like and is arranged concentrically around the inner shaft, wherein the inner shaft is fastened to the inner surface of the polymer carrier. The inner shaft is thus sheathed in a portion by the polymer carrier. The assembly of the balloon catheter and fastening of the coated polymer carrier can thus be further simplified, since the polymer carrier can be slid easily over the inner shaft of the balloon catheter. The application of the polymer carrier to the inner shaft can preferably be simplified in that the sleeve-like polymer carrier is slotted in the direction of its longitudinal axis over its entire length or part of its entire length. The polymer carrier by way of example can be welded jointly to the inner shaft with the balloon catheter after having been slid on.

In a preferred embodiment of the invention the polymer carrier is manufactured from a material which contains polyimide and/or LCP. A balloon catheter with a polymer carrier made of one of these resistant materials can be manufactured particularly easily, since they can be welded particularly effectively to the inner shaft. The polymer carrier preferably has a thickness between 10 μm and 50 μm, particularly preferably between 20 μm and 30 μm. Since the polymer carrier serves merely as carrier material for the marker, the polymer material is selected to be as thin as possible. The polymer carrier, however, must have a sufficient thickness so as not to be damaged during the production or during the use.

It is also advantageous to use at least one material of the group comprising gold, tantalum, niobium, platinum and alloys of these materials as radiopaque material. Gold in particular has a particularly high visibility under x-ray, can be processed in an excellent manner, and is biocompatible to the greatest possible extent. The coating can have a thickness between 20 μm and 100 μm, in particular between 40 μm and 60 μm. A marker applied by coating, in particular if said marker is arranged in a recess formed in the polymer carrier, can have a greater thickness than a marker attached using alternative methods, whereby an improved visibility of the balloon catheter in the x-ray image is provided.

The above object is also achieved by a method for producing a balloon catheter of this type having the features of claim 8.

In accordance with the invention, the method according to the invention includes the following steps:
providing a polymer carrier board, a polymer carrier strip, or a polymer carrier sleeve,
applying and/or introducing a coating made of a radiopaque material in the form of at least one marker to and/or into the polymer carrier board, the polymer carrier strip, or the polymer carrier sleeve, and
fastening the coated polymer carrier board, the coated polymer carrier strip, or the coated polymer carrier sleeve on the inner shaft of the balloon catheter.

Balloon catheters having the above advantages can be produced easily and economically by means of the method according to the invention.

In a preferred embodiment of the invention, in order to fasten to the inner shaft a polymer carrier strip separated from a polymer carrier board, for example a printed board comprising polyimide and/or LCP, the polymer carrier strip is firstly fixed on the inner shaft, then the inner shaft is arranged within the balloon, and then the balloon, inner shaft and polymer strip are welded to one another simultaneously at their distal end. The balloon, inner shaft and polymer carrier strip are preferably welded to one another simultaneously at their distal end, for example by means of laser, in such a way that the connection between balloon and inner shaft is fluid-tight. A catheter tip can also be welded simultaneously at the time of this welding. In order to temporarily fasten the components in the distal region (balloon, inner shaft, tip as applicable, polymer carrier strip), a heat-shrink tubing is drawn over the components and is removed again after the welding.

At the proximal end of the polymer carrier strip, this is fastened to the balloon catheter by means of welding at individual points, simultaneously to the welding of inner shaft and outer shaft, for example by means of laser. The balloon is connected in a fluid-tight manner at its proximal end to the outer shaft by means of welding, for example by means of laser.

The coating on and/or in the polymer carrier can be provided preferably by means of PVD (physical vapour deposition) or CVD (chemical vapour deposition). Furthermore, methods for deposition from the liquid phase are also widespread. Such coating methods are known for example from the manufacturing techniques in the electronics industry. Thus, commercial apparatuses exist for these techniques, which are therefore available in an economical manner. Particularly accurate coatings with a precisely adjustable composition can be provided on the printed boards using these methods.

Since the coating methods from the field of electrical engineering are used in the case of the method according to the invention for producing a balloon catheter, the entire method can be automated. This leads to much lower production costs of a balloon catheter compared to alternative methods.

In a preferred embodiment of the production method according to the invention the polymer carrier board has a longitudinal direction and a transverse direction at right angles thereto. The printed board is coated here with the radiopaque material in two strips running along the transverse direction, which strips can reach from the upper edge of the polymer carrier board to the lower edge of the polymer carrier board. The two coated strips running transversely are applied at a precisely defined distance, which corresponds to the subsequent distance between the markers. If a strip is now cut off from the coated polymer carrier board by making a cut along the longitudinal direction, this strip contains part of the first strip as first marker and, at a position at a defined distance, part of the second strip as secand marker. If further strips with the same width are cut off from the polymer carrier board, all strips are substantially identical to one another, in particular in respect of the positioning of the markers. With this method, many polymer carrier strips having two markers applied as coating can therefore be produced in an automated manner and have the same, exactly defined, reproducible distance between the markers.

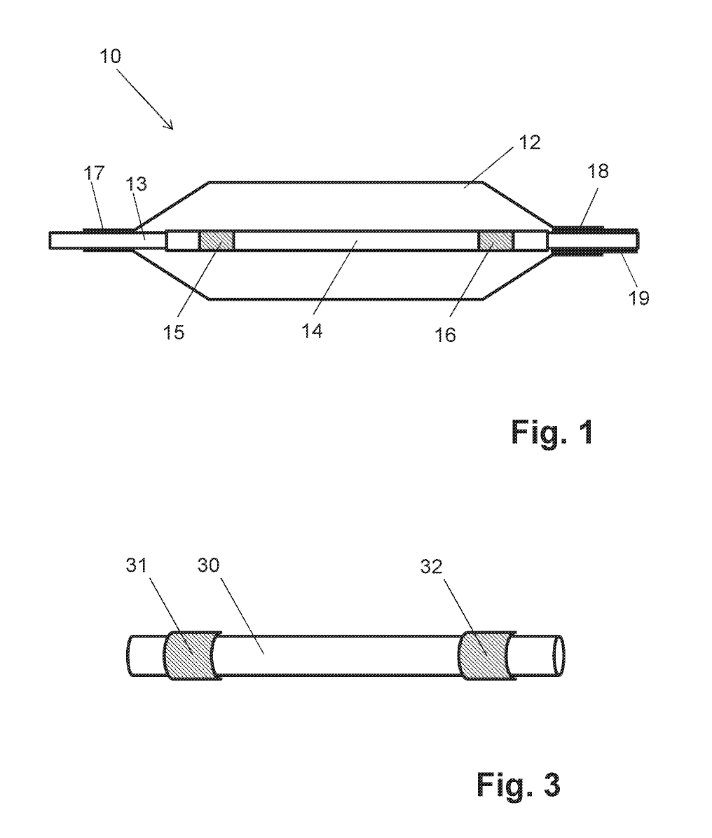

The balloon catheter 10 according to a preferred embodiment of the invention, as can be seen in FIG. 1, has an inner shaft 13 and a balloon 12, which is illustrated in a dilated state. For this purpose, the balloon was acted on by a pressure of several bar, wherein the pressure can be applied by means of a liquid or a gas. All conventional balloon materials, such as polyethylene (PET), polycarbonate (PC), polyimide (PA), PEBAX, PA11, PA12 or PVC and also blends thereof can be considered as material for the balloon.

The balloon 12 surrounds a portion of the inner shaft 13. In the surrounded portion of the inner shaft, a polymer carrier in the form of a polymer carrier strip (also referred to hereinafter as a polymer strip for short) 14 is fastened to the outer periphery of the inner shaft. A distal marker 15 and a proximal marker 16 are arranged on the polymer strip 14. Both markers 15, 16 are applied in the form of a coating to the polymer strip 14. The balloon 12 is connected in a distal region 17 to an outer shaft 19 and in a proximal region 18 to the inner shaft 13, in each case in a fluid-tight manner. The outer shaft 19 has the function of inflating and deflating the balloon 12. The balloon is therefore welded in a fluid-tight manner at its proximal end to the distal end of the outer shaft 19.

The polymer strip 14 is welded simultaneously in the distal region 17 to the balloon 12 and to the inner shaft 13 (and as applicable to a tip (not illustrated)). The polymer strip 14 is fastened in the proximal balloon region 18 at the same time as the welding, at individual points, of the inner shaft 13 and outer shaft 19. The fastening at the two ends of the polymer strip 14 is sufficient, since the polymer strip 14 is mechanically loaded only to a small extent during use of the balloon catheter.

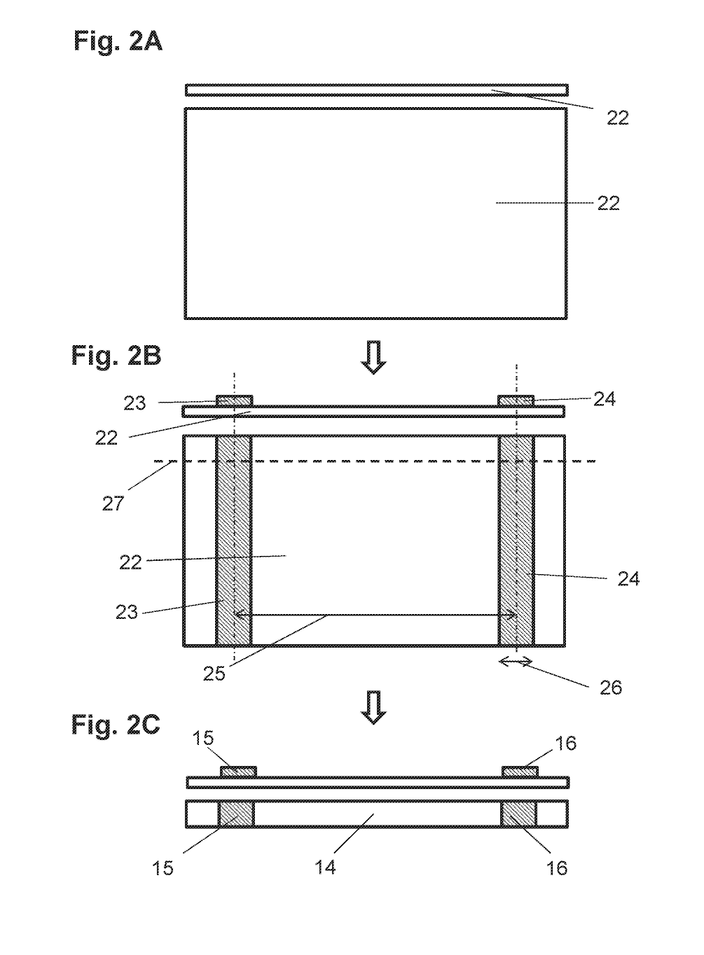

In order to produce a balloon catheter 10 according to the invention, the polymer carrier 14 with the markers 15, 16 is produced in a first step, as shown in the schematic sequence of FIGS. 2a) to 2c). For this purpose, a polymer carrier board shown in FIG. 2a, for example a printed board 22 made of LCP, is used as starting material. The thickness of the printed board 22 by way of example is 25 μm and corresponds to the thickness of the subsequent polymer strip 14. The further dimensions of the printed board 22 can correspond in a longitudinal direction to the subsequent length of the polymer strip 14 and in a transverse direction can be selected arbitrarily.

Strips 23, 24 comprising a radiopaque material are applied to this printed board 22, for example by coating methods from the gas or liquid phase, for example by means of PVD or CVD. A printed board 22 coated in this way can be seen from FIG. 2b). The thickness of the coating is for example 50 μm and corresponds to the subsequent thickness of the markers 15, 16. The strips 23, 24 run at right angles to the longitudinal direction. The strips 23, 24 have a precisely defined distance 25 (for example distance between the centrelines) for example of 15.5 mm and a width 26 for example of 1 mm, which can be set in a precise manner by the coating method. The distance 25 and the width 26 of the strips 23, 24 correspond to the subsequent distance and width of the markers 15, 16.

A plurality of individual polymer strips 14 can be cut from a printed board manufactured in this way by making cuts in the longitudinal direction. As shown in FIG. 2c), a polymer strip 14 of this type contains a region of the first strip 23 and of the second strip 24. Due to the suitable dimensioning of the printed board 22, a cut must be made merely along an individual cutting line 27 in order to separate a polymer strip from the printed board. The width of the polymer strip can be set easily by positioning of the cutting line 27 and by corresponding variation of the width of the severed strip and can be adapted to the various catheter types. It is thus possible, with a single cut, to provide the polymer strip 14 in a size that can be used for the production of a balloon catheter 10. Alternatively, other separation methods can be used in order to separate the polymer strip 14 from the printed board 22.

An alternative embodiment of the invention is shown in FIG. 3, in which, instead of the polymer strip as polymer carrier, a polymer carrier sleeve 30, for example consisting of polyimide, is used. Two markers 31, 32 in the form of a coating comprising a radiopaque material are arranged on the outer side of the polymer carrier sleeve 30. The sleeve is then preferably slotted in the direction of its longitudinal axis (i.e. longitudinally). A polymer carrier of this type embodied as a polymer carrier sleeve can be slid easily over an inner shaft of the balloon catheter in order to produce a balloon catheter and advantageously lies in a stable form on the inner shaft. The polymer carrier is fastened to the inner shaft in a manner similar to the fastening of the polymer strip.

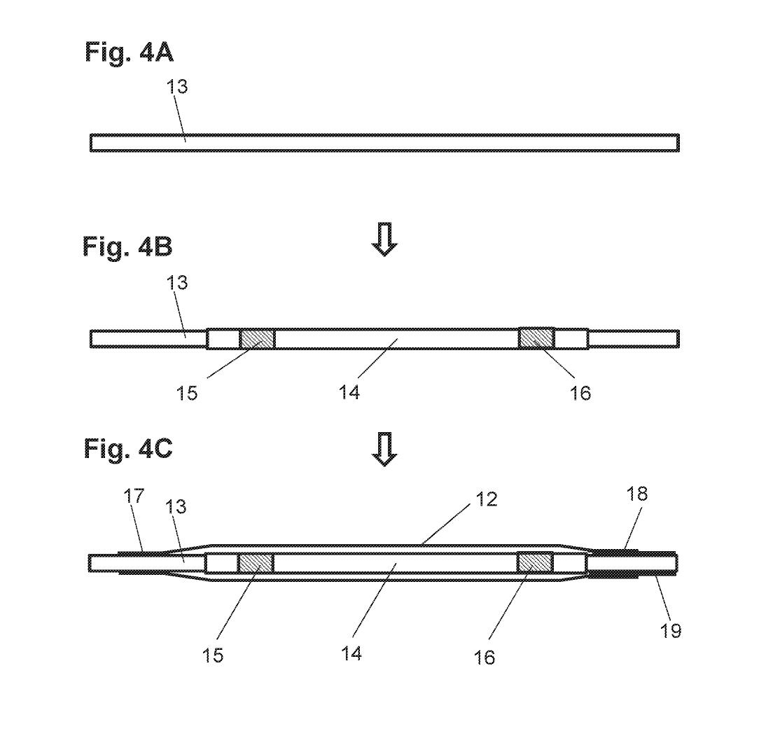

FIGS. 4a) to 4c) illustrate the production of a balloon catheter 10 according to the invention from its individual parts. Starting with the inner shaft 13 illustrated in FIG. 4a), a polymer strip 14, produced for example as explained above, comprising the markers 15, 16 is fastened to the inner shaft 13, for example by means of welding (see FIG. 4b).

In a next step illustrated in FIG. 4c), the balloon 12 is placed around the inner shaft 13 with the polymer strip 14 and is fastened to the inner shaft in the distal and proximal region 17, 18 of the inner shaft, in each case by means of a welding.

The balloon catheter 10 according to the invention produced by this method can now be inserted into a vessel. Due to the reduced rigidity of the catheter in the region of the marker compared to conventional markers, this is possible even for vessels that are difficult to access. Since the catheter according to the invention does not have any sharp edges at the markers, the risk of a perforation of the balloon by a marker is significantly reduced. The markers can also be positioned with a greater accuracy by means of the above-described production method, such that there is less scrap created from the production.

### LIST OF REFERENCE SIGNS

10 balloon catheter
12 balloon
13 inner shaft
14 polymer carrier strip, short: polymer strip
15 distal marker
16 proximal marker
17 distal region
18 proximal region
19 outer shaft
22 printed board
23 first strip
24 second strip
25 distance between the markers
26 width of the marker
27 cutting line
30 polymer carrier sleeve
31 distal marker on polymer carrier sleeve
32 proximal marker on polymer carrier sleeve

1. A balloon catheter comprising: an expandable balloon, an inner shaft arranged in the balloon, and at least one marker, wherein the at least one marker comprises a radiopaque material on and/or in a polymer carrier, the polymer carrier being fastened to the inner shaft.

2. The balloon catheter according to claim 1, comprising a first marker arranged on or in a first portion of the polymer carrier, and a second marker arranged on or in a second portion of the polymer carrier, wherein the first marker and the second marker are arranged at a predetermined distance from each other.

3. The balloon catheter according to claim 1, wherein the polymer carrier is welded at its proximal end and/or its distal end to the inner shaft.

4. The balloon catheter according to claim 1, wherein the polymer carrier adhesively bonded to the inner shaft.

5. The balloon catheter according to claim 1, wherein the polymer carrier is planar or strip-like, and curved at least in part around the inner shaft.

6. The balloon catheter according to claim 1, wherein the polymer carrier is sleeve-like.

7. The balloon catheter according to wherein the polymer carrier contains polyimide and/or LCP and/or has a thickness between 10 and 50 μm.

8. The balloon catheter according to claim 7, wherein the thickness is between 0 and 30 μm.

9. The balloon catheter according to claim 1, wherein the radiopaque material contains at least one material from the group consisting of gold, tantalum, niobium, platinum and alloys of these materials.

10. The balloon catheter according to claim 9, wherein the marker has a thickness between 20 and 100 μm.

11. The balloon catheter according to claim 10, wherein the marker has a thickness between 40 and 60 μm.

12. The balloon catheter according to claim 1, wherein the at least one marker is arranged within a recess or indentation of the polymer carrier.

13. The balloon catheter according to claim 1, wherein the radiopaque material is partially diffused into the polymer carrier.

14. The balloon catheter according to claim 1, wherein the polymer carrier is welded to the inner shaft by a peripheral, fluid-tight weld.

15. The balloon catheter according to claim 1, wherein the polymer carrier is jointly welded at a proximal end thereof to both the inner shaft and an outer shaft of the balloon catheter.

16. The balloon catheter according to claim 1, wherein the polymer carrier comprises a width that corresponds to a periphery of the inner shaft, such that the polymer carrier is curved once around the inner shaft.

17. The balloon catheter according to claim 1, wherein the polymer carrier comprises width much smaller than the periphery of the inner shaft.

18. The balloon catheter according to claim 1, wherein the polymer carrier comprises sleeve-like shape and is arranged concentrically around the inner shaft, wherein the inner shaft is fastened to the inner surface of the polymer carrier and the sheathed in a portion by the polymer carrier.

19. The balloon catheter according to claim 18, wherein the sleeve-like polymer carrier is slotted in the direction of its longitudinal axis over its entire length or part of its entire length.

20. A method for producing a balloon catheter having an expandable balloon, and an inner shaft arranged in the balloon, comprising the following steps:
providing a polymer carrier board, a polymer carrier strip, or a polymer carrier sleeve,
applying and/or introducing a radiopaque material in the form of at least one marker to and/or into the polymer carrier board, the polymer carrier strip, or the polymer carrier sleeve, and
fastening the coated polymer carrier board, the coated polymer carrier strip, or the coated polymer carrier sleeve on the inner shaft of the balloon catheter.

21. The method according to claim 20, wherein the polymer carrier strip is first fixed on the inner shaft, the inner shaft is then arranged within the balloon, and the balloon, inner shaft and polymer strip are then welded simultaneously to one another at their distal end.

22. The method according to claim 20, wherein the polymer carrier board has a longitudinal direction and a transverse direction at right angles thereto, the radiopaque material is provided in the form of at least two strips running in the transverse direction at a defined distance, and the polymer carrier strip is separated from the polymer carrier board in the longitudinal direction.

23. The method according to claim 20, wherein the radiopaque material is applied to the polymer carrier by deposition from the liquid phase or the gas phase.
